(19) United States
(12) Patent Application Publication
(10) Pub. No.: US 2012/0129923 A1
(43) Pub. Date: May 24, 2012

(54) PHARMACEUTICAL CO-CRYSTALS OF QUERCETIN

(75) Inventors: Anil Kumar Kruthiventi, Hyderabad (IN); Iqbal Javed, Hyderabad (IN); Satyanarayana Reddy Jaggavarapu, Hyderabad (IN); RaviKumar Nagalapalli, Hyderabad (IN); Ganesh Saraswatula Viswanadha, Hyderabad (IN); Solomon Kamalakaran Anand, Hyderabad (IN)

(73) Assignee: NUTRACRYST THERAPEUTICS PRIVATE LIMITED, Noida (IN)

(21) Appl. No.: 13/382,704

(22) PCT Filed: Oct. 30, 2009
(86) PCT No.: PCT/IN09/00617

§ 371 Date: Jan. 6, 2012

(30) Foreign Application Priority Data
May 20, 2009 (IN) 1154/CHE/2009

(51) Int. Cl. A61K 31/352; A61P 3/00; A61P 3/10; C07D 311/30

(52) U.S. Cl. 514/456; 549/400

(57) ABSTRACT

Disclosed herein is synergistic pharmaceutical co-crystals composition comprising Quercetin and an antidiabetic agent(s) as combination drug that have unique physical properties and biological activity which differ from the active agent in pure form. The invention further discloses process for preparation of the same and pharmaceutical compositions comprising these synergistic co-crystals.

### TECHNICAL FIELD

This invention relates to the field of pharmaceutical co-crystals of Quercetin. More particularly, the present invention relates to synergistic pharmaceutical co-crystals comprising Quercetin and an anti diabetic agent(s) as combination drug that have unique physical properties and biological activity which differ from the active agent in pure form, to process for preparation of the same and also relates to pharmaceutical compositions comprising these synergistic co-crystals.

### BACKGROUND AND PRIOR ART

Even though co-crystals were known as early as 19^{th} century, the pharmaceutical industry has recognized the potential for their applications only recently. Pharmaceutical co-crystals are crystalline molecular complexes containing therapeutic molecules. These co-crystals represent emerging class of pharmaceutical materials offering the prospects of optimized physical properties.

The application of co-crystallization can further be extended to neutral molecules, amorphous compounds, to improve their physio-chemical properties. Further, these co-crystals have utility in imparting desirable physical properties and stability, which are otherwise not achievable for the pure active agent or in combination as a simple formulation using the excipients incorporated with the active agent.

Quercetin is a plant-derived flavonoid, specifically a flavonol, used as a nutritional supplement.

The American Cancer Society says that Quercetin has been promoted as being effective against a wide variety of diseases, including cancer. As high dietary intake of fruits and vegetables is associated with reduction in cancer, and therefore scientists suspect Quercetin may be partly responsible. Quercetin is the aglycone form of a number of other flavonoid glycosides, such as rutin and quercitrin, found in citrus fruit, buckwheat and onions. Quercetin forms the glycosides quercitrin and rutin together with rhamnose and rutinose, respectively. Quercetin is classified as IARC group 3 (no evidence of carcinogenicity in humans).

Further, Quercetin is an Anti-tumor agent; induces apoptosis and inhibits synthesis of heat shock proteins. Quercetin, one of the most widely distributed flavonoids in the plant kingdom inhibits many enzyme systems including tyrosine protein kinase, phospholipase A₂, phosphodiesterases, mitochondrial ATPase, PI 3-kinase and protein kinase C; can also activate Ca²⁺ and K⁺ channels [Merck Index].

Quercetin, one of the most widely distributed flavonoids in the plant kingdom, inhibits various enzymes. This study examined its inhibitory effect on the angiotensin-converting enzyme activity through the cardiovascular response to bradykinin and angiotensin I. Quercetin pretreatment (88.7 μmol/kg p.o., 45 min; 14.7 μmol/kg i.v., 5 min) significantly potentiated the hypotensive effect of bradykinin (10 nmol/kg i.v.). This association was significantly attenuated by an antagonist of the B2 receptor. In addition, the hypertensive response to angiotensin I (0.1 nmol/kg iv) was significantly reduced by Quercetin pretreatment using the same parameters as before. These results suggest an inhibitory effect of Quercetin on the angiotensin-converting enzyme activity, similar to that of captopril. Quercetin was equally effective when given orally or intravenously. [Inhibition of Angiotensin-Converting Enzyme by Quercetin Alters the Vascular Response to Bradykinin and Angiotensin, L.P.N. Häckl, G. Cuttle, S. Sanches Dovichi, M. T. Lima-Landman, M. Nicolau, Pharmacology, 65(4), 2002].

Pre-incubation of cells with Quercetin followed by cisplatin treatment appeared to be the most effective and was correlated with strong activation of caspase-3 and inhibition of both heat shock proteins (Hsp72) and multi-drug resistance proteins (MRP) levels. The results indicate that Quercetin pretreatment sensitizes HeLa cells to cisplatin-induced apoptosis in HeLa cells [The effect of Quercetin on pro-apoptotic activity of cisplatin in HeLa cells, J. Jakubowicz-Gil et al., Biochemical Pharmacology, 69(9), 1343-1350, 2005].

The results indicate that Quercetin and rutin may be useful in the treatment of IAR and LAR in asthma via inhibition of histamine release, PLA2, and EPO, and reduced recruitment of neutrophils and eosinophils into the lung [Anti-asthmatic Action of Quercetin and Rutin in Conscious Guinea-pigs Challenged with Aerosolized Ovalbumin, Chan Hun Jung, Ji Yun Lee, Chul Hyung Cho, and Chang Jong Kim, Arch Pharm Res. 30(12), 1599-1607, 2007].

Gabriela Suchankova et al examined in HepG2 cells whether glucose-induced changes in AMP-activated protein kinase (AMPK) activity could be mediated by SIRT1, an NAD⁺-dependent histone/protein deacetylase that has been linked to the increase in longevity caused by caloric restriction. Incubation with 25 vs. 5 mM glucose for 6 h concurrently diminished the phosphorylation of AMPK (Thr 172) and ACC (Ser 79), increased lactate release, and decreased the abundance and activity of SIRT1. In contrast, incubation with pyruvate (0.1 and 1 mM) for 2 h increased AMPK phosphorylation and SIRT1 abundance and activity. The putative SIRT1 activators resveratrol and Quercetin also increased AMPK phosphorylation. None of the tested compounds (low or high glucose, pyruvate, and resveratrol) significantly altered the AMP/ATP ratio. Collectively, these findings raise the possibility that glucose-induced changes in AMPK are linked to alterations in SIRT1 abundance and activity and possibly cellular redox state [Concurrent regulation of AMP-activated protein kinase and SIRT1 in mammalian cells, Gabriela Suchankova et al., Biochemical and Biophysical Research Communications, 378(4), 836-841, 2009].

Recent findings: Quercetin bioavailability has been underestimated in the past and can be improved by food matrix components or particular delivery forms. Among the biological effects of particular relevance, the antihypertensive effects of Quercetin in humans and the improvement of endothelial function should be emphasized. Together with its antithrombotic and anti-inflammatory effects, the latter mainly mediated through the inhibition of cytokines and nitric oxide; Quercetin is a candidate for preventing obesity-related diseases. Most exiting are the findings that Quercetin enhances physical power by yet unclear mechanisms. The anti-infectious and immunomodulatory activities of Quercetin might be related to this effect [Quercetin: potentials in the prevention and therapy of disease, Bischoff, Stephan C, Current Opinion in Clinical Nutrition and Metabolic Care: 11(6), 733-740, 2008].

Quercetin increased mRNA expression of PGC-1alpha and SIRT1 (P<0.05), mtDNA (P<0.05) and cytochrome C concentration (P<0.05). These changes in mitochondrial capacity were associated with an increase in both maximal endurance capacity (P<0.05) and voluntary wheel running activity (P<0.05). These benefits of querectin on fitness without exercise training may have important implications for enhancement of athletic and military performance and may also extend to prevention and/or treatment of chronic diseases [Quercetin increases brain and muscle mitochondrial biogenesis and exercise tolerance. Am J Physiol Regul Integr Comp Physiol, 2009-3-12]

There is ample patented literature available on Quercetin. WO/2008/011364 discloses a composition containing Quercetin, vitamin B3, vitamin C, and folic acid. Also disclosed is a method of using the composition for enhancing physical or mental performance or treating various diseases or disorders.

US 20080031940 describes a composition includes 10-50 wt. % Quercetin along with papain; calcium salt; zinc salt; bee pollen; pumpkinseed; bromelain; and saw palmetto; wherein the composition is a sustained release composition in tablet or capsule form suitable for oral administration to a human. Methods of making and using the composition are provided.

Method for preventing or treating elevated blood lipid level-related diseases by administering rutin and Quercetin is disclosed in U.S. Pat. No. 6,509,372.

WO/2002/076473 describes Quercetin, its preparation and the medicinal composition containing the same and their application for preventing or treating diseases related to 5HT14 receptor or neure damage, including preventing or treating Alzeheimer's disease, drug or alcohol dependence, sleep disorders or panic state, delaying senility or improving memory function and preventing or treating neure damage caused by brain injury.

However, prior art failed to provide pharmaceutical co-crystals of Quercetin, useful in effective treatment of disease conditions. Pharmaceutical co-crystals are crystalline molecular complexes containing therapeutic molecules. These co-crystals represent emerging class of pharmaceutical materials offering the prospects of optimized physical as well as therapeutic properties.

Therefore, it is an objective of the present invention to develop synergistic pharmaceutical co-crystals of Quercetin using different active molecules as combination drugs selected from anti-diabetic agents.

### SUMMARY OF THE INVENTION

In accordance with the above objective, the present invention provides synergistic pharmaceutical co-crystals of Quercetin and anti-diabetic agents selected from biguanide group like metformin; sulfonylurea group like tolazamide, glipizide, glimepiride; Alpha-glucosidase inhibitors (acarbose); members of the thiazolidinedione class such as rosiglitazone, pioglitazone and miglitol-a glucosidase inhibitor—to act as a combination drug for metabolic syndrome associated with Hypertension, diabetic conditions inclusive of obesity and hyperlipidemia.

Accordingly in one aspect, the present invention provides the co-crystals of Quercetin which have been prepared using the above antidiabetic agents. These co-crystals showed higher solubility, dissolution rates and also found to be stable under accelerated conditions and thus suitable in preparing pharmaceutical compositions.

Accordingly in a preferred aspect of the present invention, pharmaceutical co-crystal specifically comprises of Quercetin dehydrate−Metformin as a combination drug. The co-crystal formed is further analyzed and characterized using PXRD, IR and Mass spectra. The present inventors have surprisingly noted that the melting point of the Quercetin dehydrate−Metformin co-crystal is much lower than Quercetin and higher than Metformin. Further, the solubility of Quercetin is also substantially improved when it is delivered as a co-crystal with water soluble drug like Metformin.

In another aspect the present invention provides process for preparing Quercetin dehydrate−Metformin co-crystals by hand grinding or optionally the cocrystals of the current invention can be prepared by melting method, solvent drop method using suitable solvents, followed by crystallization, if necessary.

In yet another aspect, the invention provides process for preparation of pharmaceutical co-crystals of Quercetin-antidiabetic agent wherein said process comprises providing Quercetin and an antidiabetic agent; isolating said co-crystal and incorporating it into pharmaceutical composition along with one or more suitable pharmaceutical carriers/excipients. The co-crystals of the current invention and excipients can be formulated into compositions and dosage forms according to methods known in the art.

In yet another aspect, the invention provides method for treating metabolic syndrome associated with Hypertension, diabetic conditions inclusive of obesity and hyperlipidemia, which method comprises administering ‘an effective amount’ of the ‘composition of invention’ to the subject suffering from said disorder. The subject mentioned herein is human.

In yet another aspect, the invention discloses use of the ‘composition of the invention’ in preparing the medicament intended to treat metabolic syndrome associated with Hypertension, diabetic conditions inclusive of obesity and hyperlipidemia.

### DESCRIPTION OF DRAWINGS

FIG. 1 shows the IR spectra of Metformin+Quercetin co-crystals

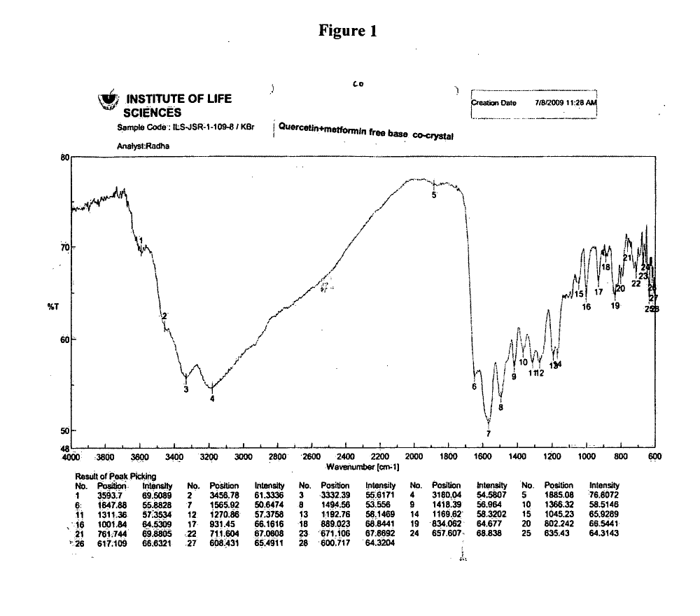

FIG. 2 shows PXRD of Metformin+Quercetin co-crystals

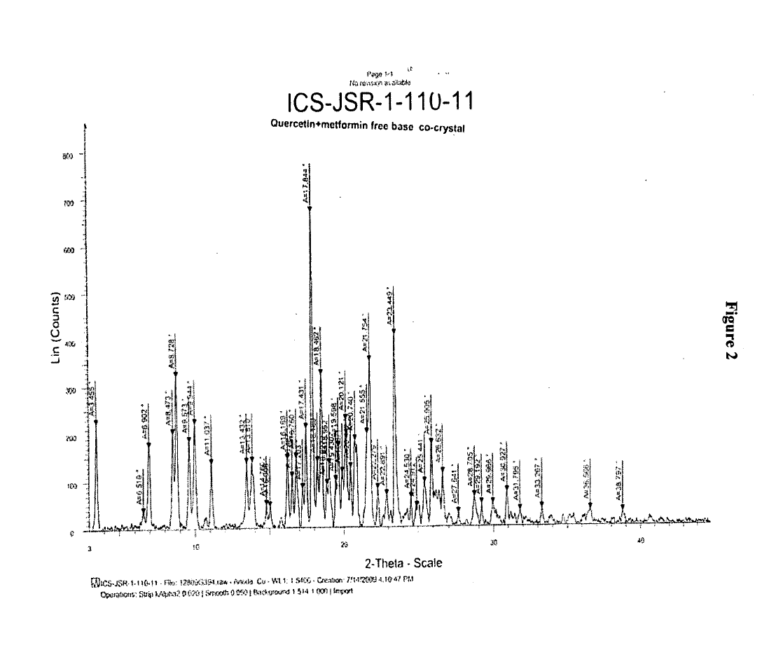

FIG. 3 shows DSC of Metformin+Quercetin co-crystals

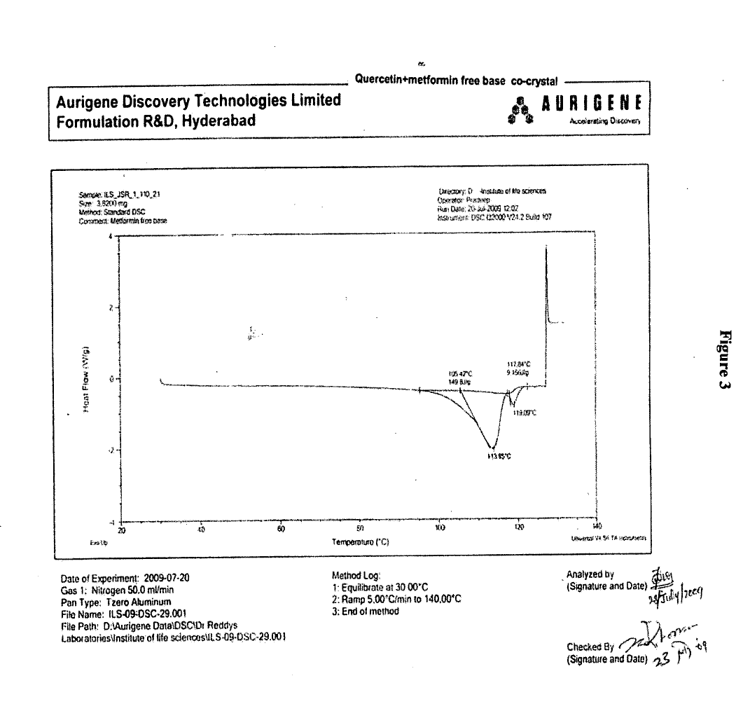

FIG. 4 shows NMR (PPM) of Metformin+Quercetin co-crystals

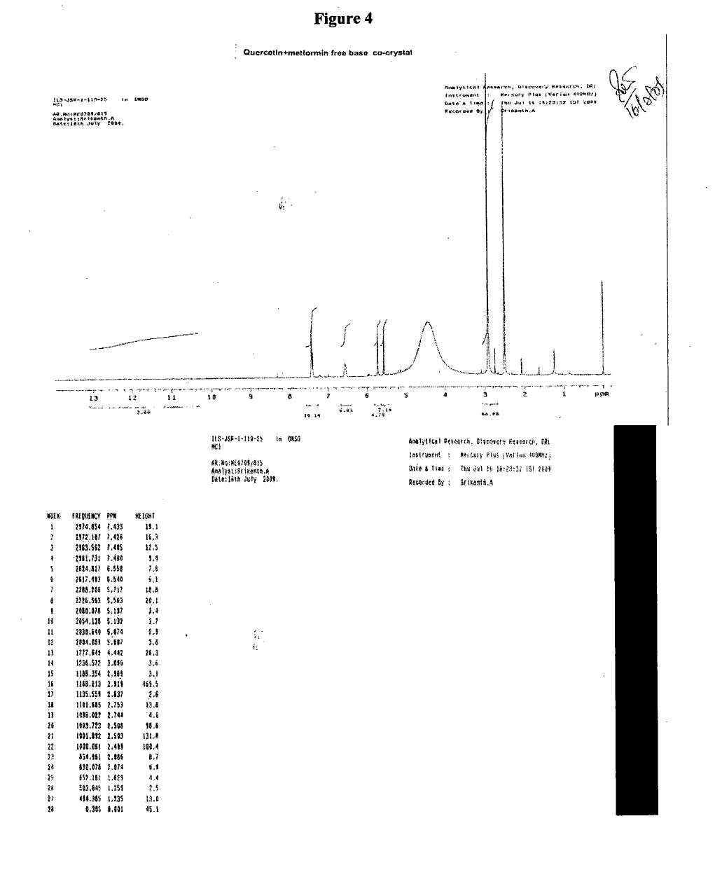

FIG. 5 shows TGA of Metformin+Quercetin co-crystal

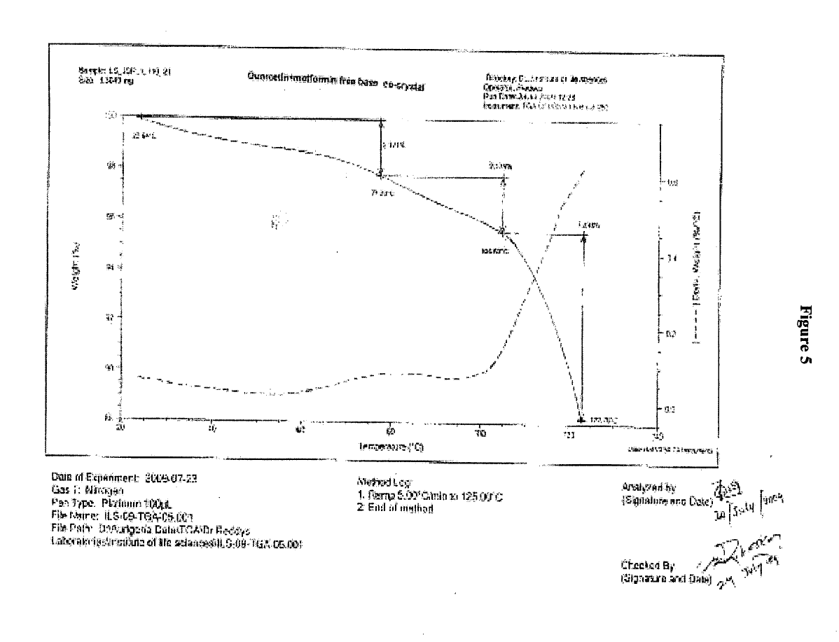

FIG. 6 shows the IR spectra of Metformin HCl+Quercetin co-crystals

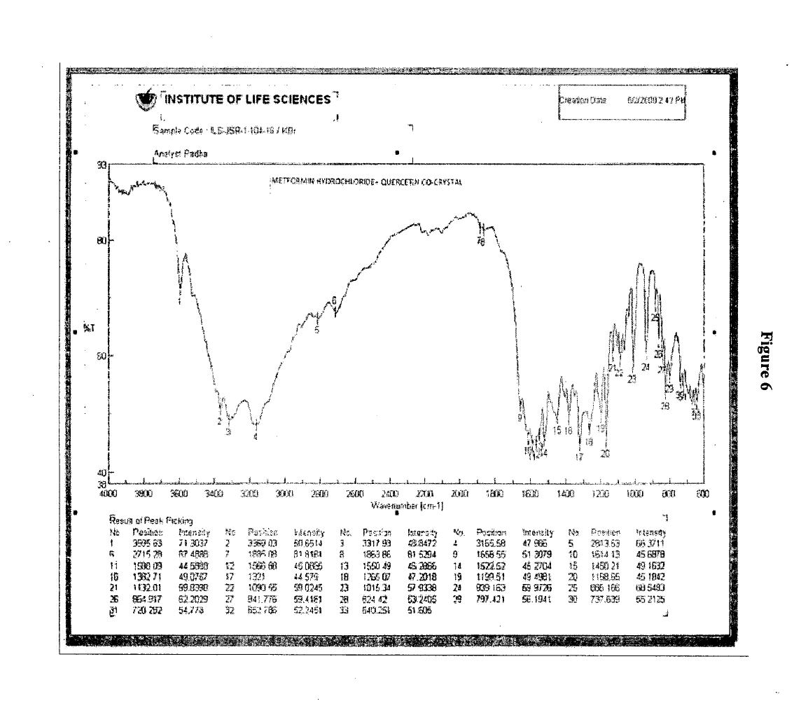

FIG. 7 shows PXRD of Metformin HCl+Quercetin co-crystals

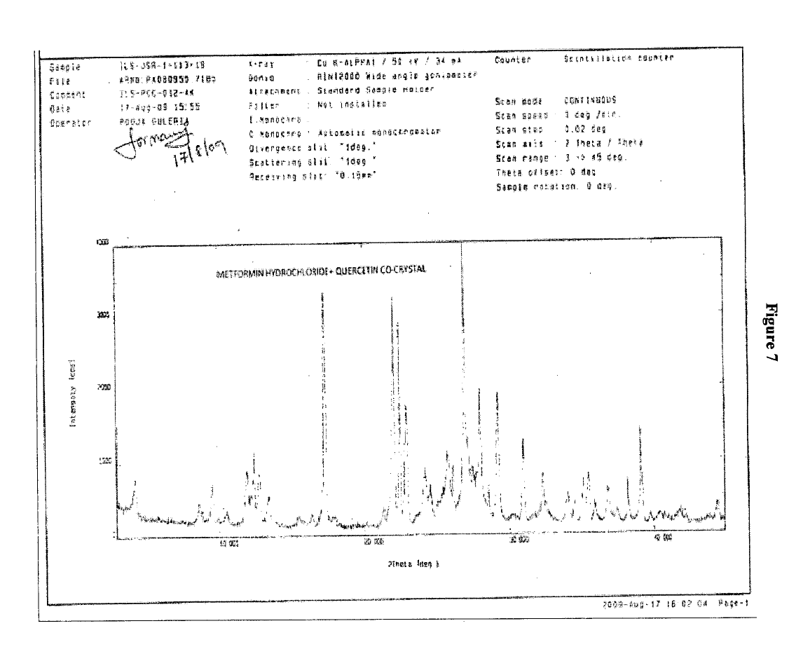

FIG. 8 shows DSC of Metformin HCl+Quercetin co-crystals

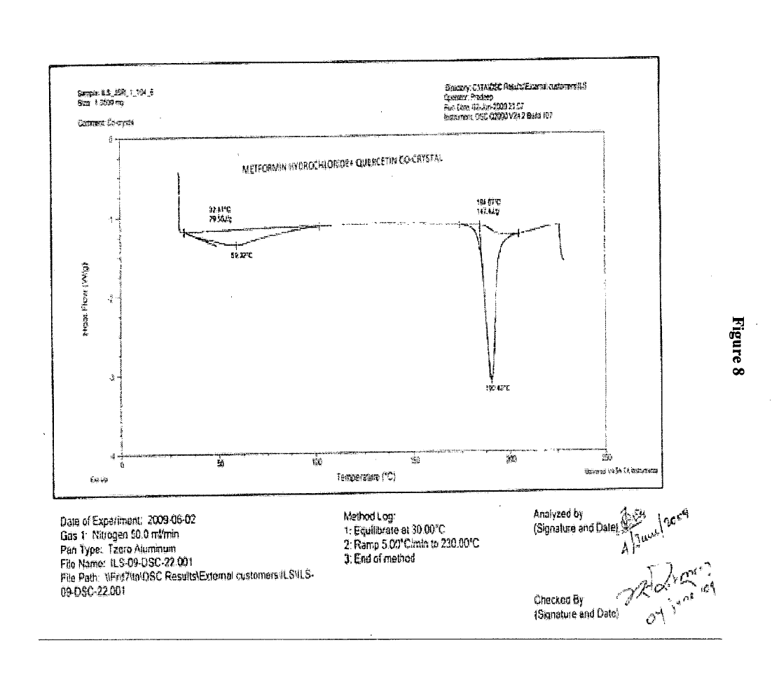

FIG. 9 shows TGA of Metformin HCl+Quercetin co-crystal

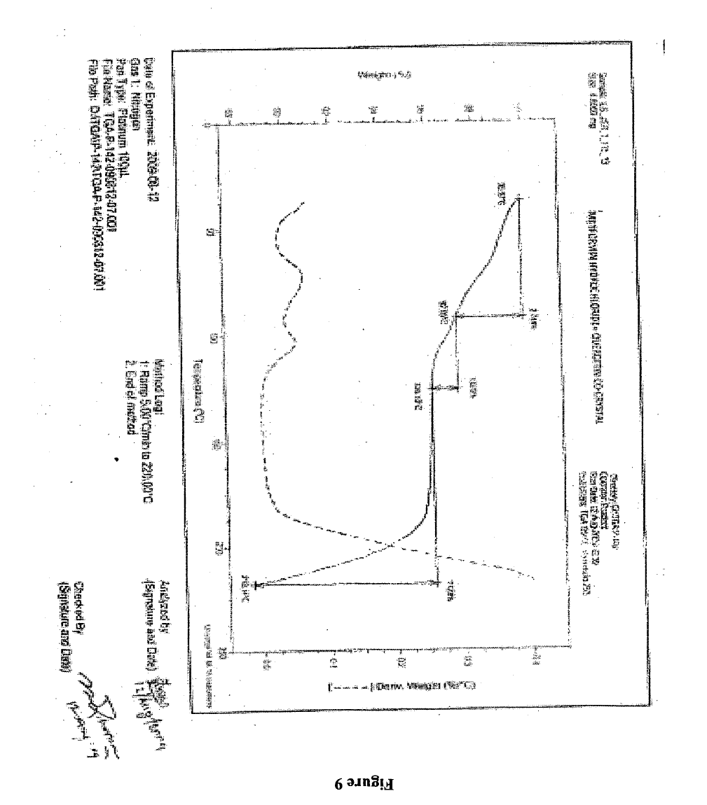

### ABBREVIATION

ILB-MCO-0904Q=Quercetin dihydrate

ILB-MCO0905Q=Quercetin dehydrate+Metformin free base co-crystal (1:2)

ILB-MCO-0906Q=Metformin hydrochloride

### DETAILED DESCRIPTION OF THE INVENTION

The invention will now be described in detail in connection with certain preferred and optional embodiments, so that various aspects thereof may be more fully understood and appreciated.

The phrase ‘composition of the invention’ herein means and includes the composition comprising the ‘co-crystals of Quercetin dehydrate-antidiabetic agent’ as described according to the present invention.

Metformin is an oral anti-diabetic drug from the biguanide class. It is the first-line drug for the treatment of type 2 diabetes, particularly in overweight and obese people and those with normal kidney function, and evidence suggests it may be the best choice for people with heart failure. It is also used in the treatment of polycystic ovary syndrome. Metformin is the only anti-diabetic drug that has been proven to protect against the cardiovascular complications of diabetes. Metformin also modestly reduces LDL and triglyceride levels.

There is ample literature available on Metformin hydrochloride and the pharmaceutical compositions comprising the same for the treatment of type II diabetes.

In this application, the inventors have achieved the pharmaceutical co-crystals of Quercetin using numerous antidiabetic agents as combination drug, which works synergistically against metabolic syndrome associated with Hypertension, diabetic conditions inclusive of obesity and hyperlipidemia, thus enhancing the efficacy of the combination even in lower doses.

Accordingly, the invention provides synergistic pharmaceutical compositions comprising the co-crystals of Quercetin and antidiabetic agent(s). The antidiabetic agent is selected from biguanide group like metformin; sulfonylurea group like tolazamide, glipizide, glimepiride; Alpha-glucosidase inhibitors (acarbose); members of the thiazolidinedione class such as rosiglitazone, pioglitazone and miglitol-a glucosidase inhibitor, to act as a combination drug for metabolic syndrome associated with Hypertension, diabetic conditions inclusive of obesity and hyperlipidemia, whereas Quercetin can be selected in the form of hydrates or its polymorphs.

Accordingly in one of the preferred embodiment, the present invention specifically describes co-crystals of Quercetin dehydrate and Metformin to act as a combination drug for metabolic syndrome associated with Hypertension, diabetic conditions inclusive of obesity and hyperlipidemia. These co-crystals showed higher solubility, dissolution rates and also found to be stable under accelerated conditions and thus suitable in preparing pharmaceutical compositions.

In another embodiment, the invention provides a process for preparation of co-crystal of Quercetin dehydrate−Metformin in the ratio of about 1:1 to about 1:3; by hand grinding. Optionally, the co-crystals of the current invention can be prepared by melting method, solvent drop method using suitable solvents, followed by crystallization, if necessary.

In one of the preferred embodiment, the process for preparation of Quercetin dehydrate and Metformin free base (1:2) co-crystal comprises of ground neating the heated Quercetin dihydrate with Metformin free base for 3 minutes using a mortar and pestle. The Metformin free base is prepared by dissolving 1:1 molar ratio of Metformin hydrochloride and sodium hydroxide in 2-propanol. Further, the process for preparation of Quercetin dehydrate and Metformin hydrochloride (1:2) co-crystal comprises neat (solventless) grinding of Metformin hydrochloride and Quercetin dehydrate for 3 minutes to make it homogeneous mixture. The resultant product so obtained was subjected to analytical studies.

X-Ray Powder Diffractometry:

Powder data were collected on PANalytical, X'Pert PRO X-ray powder diffractometer using a parallel beam of monochromated Cu—K_{α} radiation (λ=1.5418 Å) and an X'Celerator detector at 45 kV voltage and 40 mA Current. Diffraction patterns were collected over the 2θ range 3-45°.

Further, these co-crystals were characterized by thermal analysis. The inventors have surprisingly noted that the melting point of the Metformin-Quercetin dehydrate co-crystal formed is much lower than the Quercetin and higher than Metformin.

1. The Physical Characteristics of Quercetin Dehydrate−Metformin Hydrochloride (1:2) Co-Crystals are as Tabulated Below:

| Quercetin | | | | |
|---|---|---|---|---|
| dehydrate and | | | | |
| Metformin | | | | |
| HCl | Method | IR | M.P | PXRD |
| 1:2 | Neat(solventless) | slight change | 234-241° C. | Change is |
| | Grinding. | | | observed. |

| | | | Quercetin dehydrate |
|---|---|---|---|
| Characterization | | | and Metformin HCl |
| Method | Quercetin | Metformin HCl | co-crystal |
| MELTING POINT(° C.) | 300° C. | 222° C. to 226° C. | 234-241° C. |
| IR DATA: | 3590 cm⁻¹, 3409 | 3370 cm⁻¹, 3298 | 3391 cm⁻¹, 3368 cm⁻¹, |
| | cm⁻¹, 3319 cm⁻¹, | cm⁻¹, 3168 cm⁻¹, | 3292 cm⁻¹, 3162 cm⁻¹, |
| | 1664 cm⁻¹, | 2814 cm⁻¹, 2698 | 2882 cm⁻¹, |
| | 1613 cm⁻¹ | cm⁻¹, 1630 cm⁻ | 1653 cm⁻¹, 1617 cm⁻¹, |
| | 1561 cm⁻¹, 1522 cm⁻¹, | 1575 cm⁻¹, 1482 | 1588 cm⁻¹, 1509 cm⁻¹, |
| | 1321 cm⁻¹, | cm⁻¹, 1065 cm⁻¹ | 1362 cm⁻¹, 1320 cm⁻¹ |
| | 1169 cm⁻¹, 1015 cm⁻¹ | | 1065 cm⁻¹ |
| PXRD DATA (2Theta): | 10.500, 11.30, 11.46, | 12.238, 12.394, | 4.489, 9.719, 12.65, 13.003, |
| | 11.86, 12.16 | 12.905, 17.855, | 17.575, 22.26, 23.155 25.98, |
| | 14.42, 15.56, 27.14 | 18.522, 21.831, | 28.20, 29.400 |
| | | 22.531, 23.421, | |
| | | 24.713, 26.604, | |
| | | 29.681, 32.7513 | |
| DSC | Sharp peak | Sharp peak | Broad peak observed at |
| | observed at | observed at | 190° C. |
| | 320° C. | 227° C. | |

2. The Physical Characteristics of Quercetin Dehydrate−Metformin (1:2) Co-Crystals are as Tabulated Below:

| | | | Quercetin dehydrate |
|---|---|---|---|
| CHARACTERISATION | Quercetin | | and Metformin co- |
| METHODS | (150° C. heated) | Metformin | crystal |
| IR(cm⁻¹) | 3312 cm⁻¹, 1668 | 3368 cm⁻¹, 3297 | 3456 cm⁻¹, 3332 cm⁻¹, |
| | cm⁻¹, 1617 cm⁻¹, | cm⁻¹, 3179 cm⁻¹, | 3180 cm⁻¹, 1647 cm⁻¹, |
| | 1560 cm⁻¹, 1513 cm⁻¹ | 1635 cm⁻¹, | 1565 cm⁻¹, 1494 cm⁻¹, |
| | 1357 cm⁻¹, | 1619 cm⁻¹, | 1418 cm⁻¹, 1311 cm⁻¹, |
| | 1163 cm⁻¹, 1093 cm⁻¹ | 1059 cm⁻¹ | 1192 cm⁻¹, 1045 cm⁻¹ |
| PXRD(2 THETA) | 4.88, 8.69, 10.89, | 12.13, 12.74, | 3.45, 6.90, 8.47, 8.72, |
| | 11.29, 12.55, 14.43, | 15.74, 16.29, | 9.57, 9.94, 13.43, 13.81, |
| | 26.95, 27.55 | 17.78, 18.24, | 16.16, 16.76, 17.43, |
| | | 22.73, 23.16, | 17.84, 18.46, 19.58, |
| | | 24.41, 25.89, | 20.74, 21.75, 23.44, |
| | | 27.73, 29.20 | 25.90 |
| MELTING POINT(° C.) | 320° C. | 101-105° C. | 120-129° C. |
| DSC | Sharp peak | Broad peak | Broad peak observed at |
| | observed at | observed at | 119° C. |
| | 320° C. | 111° C. | |
| NMR(PPM) | δ12.492(1H,S), | δ2.931(6H,S), | δ 2.931(6H,S) |
| | δ10.775(1H,S), | δ6.743(4H,S) | δ 7.67(1H,d), |
| | δ9.58(1H,S), δ9.35 | δ7.213(2H,S) | δ 7.68(1H,d), |
| | (1H,S) δ9.29(1H,S), | | δ 6.89(1H,d) |
| | δ7.67(1H,d), | | δ 6.87(1H,S), |
| | δ 7.68(1H,d), δ | | δ 6.18(1H,S) |
| | 6.89(1H,d) | | |
| | δ 6.87(1H,S), δ | | |
| | 6.18(1H,S | | |
| TLC in 50% EtOAc + | 0.6 | 0.0 | 0.0 & 0.6 |
| Hexane | | (doesn't move | |
| R_{f} | | in TLC) | |
| | | Presence | |
| | | indicated by | |
| | | ninhydrin | |
| TGA | No weight loss | Weight loss | At 105.6° C. two water |
| | observed below | observed at | molecule weight loss |
| | 300° C. | 147° C. is around | was observed and at |
| | | 46 mass | 248.14° C. & 144.9 |
| | | | weight loss observed |

Dissolution and Solubility:

The compounds of the present invention are finely grinded and passed through standard mesh filter and particles with 75-180 micron are tested for their solubility and dissolution profile.

According to the present invention, the pharmaceutical co-crystals of Quercetin dehydrate and Metformin have showed higher dissolution rates when compared to the parent molecules and were also found to be stable under accelerated conditions. Further, the solubility of the Quercetin dehydrate—Metformin co-crystals prepared according to the present invention when compared with the parent molecule was found to be greater than the parent molecule, i.e. Quercetin (a highly insoluble molecule).

Stability:

The stability of the Quercetin dehydrate−Metformin co-crystal was monitored, once in seven days for 3 months. The co-crystal were found to be stable under desiccated conditions, however, co-crystal tends to be hygroscopic under normal conditions.

In another embodiment, the invention provides process for preparation of a pharmaceutical Quercetin dehydrate-antidiabetic co-crystal wherein said process comprises providing Quercetin with at least one of the antidiabetic agent; isolating said co-crystal and incorporating it into pharmaceutical composition along with one or more suitable pharmaceutical carriers/excipients. The co-crystals of the invention are found to be more efficacious than the API, as seen from the Animal studies. Further, the pharmaceutical composition of the invention may be any pharmaceutical form which maintains the crystalline form of a co-crystal of the invention.

The pharmaceutical composition may be a solid form, a liquid suspension or an injectable composition. The active ingredient (s) and excipients can be formulated into compositions and dosage forms according to methods known in the art.

The ‘composition of the invention’ is preferably administered along with one or more pharmaceutical excipient(s)/carrier(s). The oral administration may be accomplished by ingesting the composition preferably in a form of tablet/capsule/liquid with a glass of water. The other dosage forms like hard gelatin capsules, powders, liquid capsules, syrups, suspensions, elixirs are also equally good modes of oral administration.

The quantity of the compound used in pharmaceutical co-crystal compositions of the present invention will vary depending upon the body weight of the patient and the mode of administration and can be of any effective amount to achieve the desired therapeutic effect.

In yet another embodiment, the invention provides method for treating metabolic syndrome associated with Hypertension, diabetic condition inclusive of obesity and hyperlipidemia; which method comprises administering ‘an effective amount’ of the ‘composition of invention’ to the subject suffering from said disorder. The subject mentioned herein is human.

The ‘effective amount’ as described above means and includes the amount required to treat/alleviate the severity of symptoms associated with this ailments as decided by the persons of ordinary skill in the art.

In yet another embodiment, the invention discloses use of the ‘composition of the invention’ in preparing the medicament intended to treat metabolic syndrome associated with Hypertension, diabetic condition inclusive of obesity and hyperlipidemia.

Anti-Hypertensive, Anti-Diabetic Activity Inclusive of Obesity and Hyperlipidemia:

The pharmaceutical co-crystals of Quercetin dehydrate−Metformin are tested for its anti-diabetic activity inclusive of obesity and hyperlipidemia as well as for hypertension by (i) determining the acute and chronic plasma glucose lowering activity of test compound in db/db mice and (ii) determining the chronic effect of test compounds on plasma insulin, triglyceride, cholesterol and body weight.

Methods:

Db/db mice were acclimatized to dosing and exposed to tail cut and r.o.p bleeding. Animals were grouped based on plasma glucose, triglyceride and cholesterol values. Acute effect of test compound on plasma glucose levels determined 3 hrs post dosing followed by administration of test compound for 21 days. After 14 days treatment, 4 hrs fasting plasma glucose levels were measured. After 21 days treatment, 4 hrs fasting plasma glucose levels were measured followed by test compound administration and measurement of plasma glucose levels 3 hrs post dosing.

Results:
Acute administration of ILB-MCO-0905Q and ILB-MCO-0906Q showed significant reduction in plasma glucose levels 3 hrs post dosing
After 21 days treatment test compounds did not show significant reduction in 4 hrs fasting plasma glucose levels. ILB-MCO-0905Q showed significant reduction in plasma insulin values compared to control group.
After 21 days treatment followed by acute administration of ILB-MCO-0905Q and ILB-MCO-0906Q, caused significant reduction in plasma glucose levels 3 hrs post dosing.

The results described herein above, conclusively proves the introduction of synergy in the co-crystals by combining the antidiabetics with the said co-crystal former, which act not only as co-crystal formers but also contribute to enhance the efficacy of the anti-diabetic agent by lowering the levels of plasma glucose, plasma insulin, triglyceride, cholesterol; improving the body weight and lowering the blood pressure, when compared to the administration of Quercetin and Metformin alone.

It will be evident to those skilled in the art that the invention is not limited to the details of the foregoing illustrative examples and that the present invention may be embodied in other specific forms without departing from the essential attributes thereof, and it is therefore desired that the present embodiments and examples be considered in all respects as illustrative and not restrictive, reference being made to the appended claims, rather than to the foregoing description, and all changes which come within the meaning and range of equivalency of the claims are therefore intended to be embraced therein.

### EXAMPLES

### Example 1

### Preparation of Metformin from Metformin Hydrochloride

1:1 molar ratio of Metformin hydrochloride and sodium hydroxide were dissolved in 2-Propanol. The suspension was stirred for 3 hours at 313K, filtered and the filtrate evaporated to yield a white solid free of chloride ion (checked with 0.1 M AgNO3 solution). The resultant product was confirmed as Metformin using IR, NMR studies and melting point (111° C.).

Preparation of Metformin Free Base and Quercetin Dehydrate Co-Crystal

Quercetin dihydrate (heated to 150° C., 30.3 mg, and 0.1 mmol) and Metformin free base (25.8 mg, 0.2 mmol) were neat ground for 3 mins using a mortar and pestle. The resultant solid was subjected to analytical studies.

### Example 2

### Preparation of Quercetin Dehydrate and Metformin Hydrochloride Co-Crystal

Quercetin dihydrate (heated to 150° C., 30.3 mg, and 0.1 mmol) and Metformin hydrochloride (25.8 mg, 0.2 mmol) were neat ground for 3 mins using a mortar and pestle. The resultant solid was subjected to analytical studies.

Optionally, the cocrystals of the current invention can be prepared by melting method or solvent drop method using suitable solvents, followed by crystallization, if necessary.

The formation of these co-crystal or salt was confirmed by powder X-ray powder diffractometry and IR spectroscopy.

### Example 4

### Solubility Studies for Co-Crystals and Salts of the Present Invention

The solubility studies for co-crystals and salts were performed according to Higuchi and Connor's method with some variations. Excess amounts (100 mg) of the samples were suspended in 10 mL of water in round bottom flask. Solutions were stirred at 300 rpm using a magnetic stirrer. After 72 h, the suspensions were filtered through a paper filter (Whatman 40)) and filtered aliquots were sufficiently diluted, the absorbance of the samples were measured at 232 nm and the values were normalized for API. Finally, the concentration of API after 72 h (apparent aqueous solubility) in each sample was determined from the previously made standard graph. A standard graph was made by measuring the absorbance of varied concentrations of API in water using a UV spectrophotometer (Nanodrop UV/vis spectrometer) at λₘₐₓ 232 nm. (Refer FIGS. 10-12)

### Example 5

### Dissolution Profile of the Co-Crystals of the Present Invention

Co-crystal taken (2 mole) in 50 ml round bottom flask and 50 ml of nano pure water added over it. This solution was stirred at 300 rpm. 5 ml of solution was taken out at 5, 10, 30, 60 and 120 minutes interval and filtered through Whatman 40 filter paper. The filtrate was diluted by 10 times and UV absorption was measured.

### Example 6

### Clinical Study of the Co-Crystals of the Present Invention

Anti-diabetic effect of ILB-MCO-0904Q (Quercetin dihydrate), ILB-MCO0905Q (Co-crystal of Quercetin dehydrate and Metformin Free base (1:2)) & ILB-MCO-0906Q (Metformin hydrochloride) in db/db mice:

Principle of Test:

Primary Objective:

To determine the acute and chronic plasma glucose lowering activity of test compound in db/db mice.

Secondary Objective:

To determine the chronic effect of test compounds on plasma insulin, triglyceride, cholesterol and body weight.

Material and Methods:

Animals: In house colony db/db mice

Age: 7 weeks Sex: Male

Kits: Plasma glucose, cholesterol and triglyceride were measured using Dade Behring auto analyzer. Plasma insulin values measured using Rat/mouse insulin ELISA kit (Millipore, lot no 16006280). Tail cut method plasma glucose values measured using Contour TS glucometer strips (Lot WK8MD3E32A).

Compounds and Vehicle: All test-compounds were formulated in tween CMC (10% of 2.5% tween 80+90% of 0.25 CMC) and prepared daily about 1 hr before administration. Animals were dosed for 21 days through oral gavage at dose volume of 10 ml/kg body weight.

Groups:

Group 1: Vehicle control (n=10), 10 ml/kg, once daily, per oral

Group 2: Compound ILB-MCO-0904Q (n=10), 300 mg/kg, once daily, per oral

Group 3: Compound ILB-MCO-0905Q (n=10), 300 mg/kg, once daily, per oral

Group 4: Compound ILB-MCO-0906Q (n=10), 300 mg/kg, once daily, per oral

Experimental Design:

db/db mice 8-9 weeks age

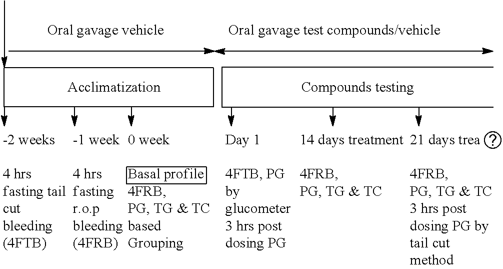

r.o.p: retero orbital plexus

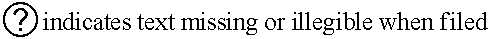

Experimental Details:

Male db/db mice, (bred in-house) aged around 7 weeks at the time of initiation of acclimatization, were used for the study. These mice, (5 per cage), were housed under standard temperature, light & humidity conditions and provided with NIN pellet feed and water, ad libitum.

During acclimatization period till the basal measurements, each mouse was orally dosed daily once with 0.4 ml of vehicle. At the end of first week of acclimatization animals were exposed to the stress of 4 hrs fasting, (feed removed at 7 AM.) and bleeding (at 11.00 AM morning), by tail cut method (side wise, small cut with scalpel blade 1 cm. above the tail end). After a gap of 2 days animals were exposed to stress of bleeding via retro-orbital plexus (r.o.p) after 4 hrs fasting. Basal plasma profile measurement (PG, TG & TC), in 4 hrs fasted mice, was conducted at the end of 2nd week of acclimatization. Animals were grouped based on basal plasma glucose, triglyceride levels and cholesterol levels. Plasma samples were frozen for insulin measurement.

ACUTE EFFECT MEASUREMENT: On the day of initiating study animals were fasted for 4 hrs and plasma glucose was measured with Glucometer by tail cut method. Animals were dosed with test compound/vehicle according to their body weight and 3 hrs post dosing plasma glucose levels were measured with Glucometer. Immediately animals were provided with measured amount of feed.

CHRONIC EFFECT: Animals were dosed daily at around 10 AM to 11 AM with corresponding test compounds/vehicle at a volume of 10 ml/kg of body weight. Body weight, water intake and food intake was measured daily. After 14 days treatment plasma profile (PG, TG & TC) was measured in 4 hrs fasted animals. On that day animals were dosed post bleeding after keeping feed at 4 PM. After 21 days treatment plasma profile (PG, TG & TC) was measured in 4 hrs fasted animals. After bleeding, animals were administered with test compounds/vehicle and plasma glucose was measured 3 hrs post dosing with Glucometer. Immediately animals were provided with feed.

Blood samples from r.o.p bleeding were collected in micro centrifuge tubes containing 5 ul of EDTA Na (200 ng/ml). Plasma separated by centrifuging blood samples at 6000 rpm at 40 C for 5 minutes. Plasma glucose, total cholesterol and triglyceride were measured using Dade Bhering auto analyser. Plasma insulin was measured (in duplicate samples), using Millipore mouse/rat insulin ELISA kit.

Calculation & Statistics:

Acute effect percent reduction in PG was calculated for each animal using the formula

{(0 hr PG−test hr PG)/0 hr PG}×100

The percent reduction in plasma profile parameters were calculated using the formula 1−(tt/tc)/bt/bc)×100; where tt: test day treated group mean value of the parameter; tc: test day control group mean value of the parameter; bc: Basal Control group mean value; bt: Basal treated group mean value.

Statistics were applied using One-Way ANOVA followed by Dunnett's test using sigma stat software. The values shown in the tables are Mean±SEM

Results:

Acute Effect:

TABLE 1
Acute effect of test compounds on plasma glucose levels in d/b/db
mice (mg/dl; Mean ± SEM)

| | | | % |
|---|---|---|---|
| Group | 0 Hr | 3 Hr | Reduction |
| Vehicle Control | 377 ± 44 | 290 ± 39 | 23 ± 4 |
| (n = 10) | | | |
| ILB-MCO-0904Q | 398 ± 67 | 293 ± 65 | 31 ± 4 |
| 300 mg/kg (n = 10) | | | |
| ILB-MCO-0905Q | 358 ± 34 | 186 ± 21 | 48 ± 4* |
| 300 mg/kg (n = 10) | | | |
| ILB-MCO-0906Q | 373 ± 43 | 181 ± 25 | 52 ± 4* |
| 300 mg/kg (n = 10) | | | |

*P < 0.05 vs control group

Chronic Effect: Post 4 Hr Fasting, Pre Dosing, 14 Days

TABLE 2
Chronic effect of test compounds on plasma glucose levels
(mg/dl; Mean ± SEM) before dosing on the day of bleeding

| | Basal | 14th day | % | Basal | 14th day | % | Basal | 14th day | % |
|---|---|---|---|---|---|---|---|---|---|
| Group | PG | PG | Red | TC | TC | Red | TG | TG | Red |
| Vehicle Control | 395.36 ± | 449.24 ± | — | 155.90 ± | 153.02 ± | — | 146.70 ± | 149.08 ± | — |
| (n = 10) | 31.93 | 37.50 | | 5.03 | 4.41 | | 8.49 | 11.31 | |
| ILB-MCO-0904Q | 402.50 ± | 476.41 ± | −4 | 156.25 ± | 154.43 ± | −1 | 138.00 ± | 129.08 ± | 8 |
| 300 mg/kg | 26.82 | 45.57 | | 4.53 | 6.50 | | 8.04 | 5.61 | |
| (n = 10) | | | | | | | | | |
| ILB-MCO-0905Q | 399.40 ± | 417.71 ± | 8 | 160.42 ± | 153.68 ± | 2 | 135.94 ± | 126.70 ± | 8 |
| 300 mg/kg | 26.54 | 37.85 | | 3.77 | 3.49 | | 6.92 | 6.25 | |
| (n = 10) | | | | | | | | | |
| ILB-MCO-0906Q | 402.43 ± | 443.33 ± | 3 | 155.90 ± | 144.51 ± | 6 | 136.10 ± | 114.11 ± | 17 |
| 300 mg/kg | 26.13 | 40.27 | | 5.08 | 4.61 | | 5.39 | 4.76 | |
| (n = 10) | | | | | | | | | |

Chronic Effect: Post 4Hr Fasting, Predosing, 21 Days

TABLE 3
Chronic effect of test compounds on plasma glucose levels
(mg/dl; Mean ± SEM) before dosing on the day of bleeding

| | Basal | 21st day | % | Basal | 21st day | % | Basal | 21st day | % |
|---|---|---|---|---|---|---|---|---|---|
| Group | PG | PG | Red | TC | TC | Red | TG | TG | Red |
| Vehicle Control | 395.36 ± | 425.01 ± | — | 155.90 ± | 178.20 ± | — | 146.70 ± | 190.52 ± | — |
| (n = 9) | 31.93 | 42.71 | | 5.03 | 24.04 | | 8.49 | 25.66 | |
| ILB-MCO-0904Q | 402.50 ± | 432.69 ± | 0 | 156.25 ± | 151.25 ± | 15 | 138.00 ± | 141.99 ± | 21 |
| 300 mg/kg | 26.82 | 42.38 | | 4.53 | 4.93 | | 8.04 | 8.25 | |
| (n = 10) | | | | | | | | | |
| ILB-MCO-0905Q | 399.40 ± | 368.96 ± | 14 | 160.42 ± | 160.25 ± | 13 | 135.94 ± | 129.66 ± | 27 |
| 300 mg/kg | 26.54 | 32.95 | | 3.77 | 4.29 | | 6.92 | 5.38 | |
| (n = 10) | | | | | | | | | |
| ILB-MCO-0906Q | 402.43 ± | 400.80 ± | 7 | 155.90 ± | 157.86 ± | 11 | 136.10 ± | 127.00 ± | 28 |
| 300 mg/kg | 26.13 | 42.77 | | 5.08 | 8.76 | | 5.39 | 5.65 | |
| (n = 10) | | | | | | | | | |

Chronic Effect: 3 Hr Post Dosing, 21 Days

TABLE 4
Chronic effect (21 days) of test compounds on plasma glucose
3 hrs post dosing (mg/dl; Mean ± SEM)

| | 21 thday | |
|---|---|---|
| | 3 hr post | |
| Group | dosing PG | % Red |
| Vehicle Control | 360 ± 47 | — |
| (n = 9) | | |
| ILB-MCO-0904Q | 345 ± 41 | 4 |
| 300 mg/kg (n = 10) | | |
| ILB-MCO-0905Q | 225 ± 33* | 38 |
| 300 mg/kg (n = 10) | | |
| ILB-MCO-0906Q | 200 ± 31* | 44 |
| 300 mg/kg (n = 10) | | |

*P < 0.05 vs control group

Chronic Effect: Post 4 Hr Fasting, Predosing, 21 Days Treatment

TABLE 5
Chronic effect of test compounds on plasma insulin levels
(ng/ml; Mean ± SEM)

| | Basal | 21thday | % | % Reduc- |
|---|---|---|---|---|
| Group | insulin | insulin | Reduction | tion@ |
| Vehicle Control | 3.90 ± 0.38 | 4.69 ± 0.77 | −21.81 ± 19.11 | — |
| (n = 9) | | | | |
| ILB-MCO-0904Q | 2.94 ± 0.20 | 2.90 ± 0.33 | 0.29 ± 9.87 | 18 |
| 300 mg/kg (n = 10) | | | | |
| ILB-MCO-0905Q | 4.55 ± 0.50 | 3.03 ± 0.42 | 30.56 ± 8.10* | 45 |
| 300 mg/kg (n = 10) | | | | |
| ILB-MCO-0906Q | 4.42 ± 0.63 | 3.82 ± 0.72 | 15.32 ± 7.16 | 28 |
| 300 mg/kg (n = 10) | | | | |

*P < 0.05 vs control group
@ calculated after adjusting for control group change

TABLE 6
Chronic effect of test compounds on body weight (mg; Mean ± SEM)

| | Basal | Final day | |
|---|---|---|---|
| Group | Body weight | Body weight | % Increase |
| Vehicle Control | 39.1 ± 1.2 | 44.1 ± 1.1 | 11.2 ± 2.3 |
| (n = 9) | | | |
| ILB-MCO-0904Q | 39.1 ± 1.2 | 44.6 ± 1.3 | 12.2 ± 2.3 |
| 300 mg/kg (n = 10) | | | |
| ILB-MCO-0905Q | 39.2 ± 0.8 | 43.7 ± 1.0 | 10.1 ± 2.5 |
| 300 mg/kg (n = 10) | | | |
| ILB-MCO-0906Q | 38.5 ± 0.5 | 45.1 ± 0.6 | 14.5 ± 0.9 |
| 300 mg/kg (n = 10) | | | |

Summary of test compound effects in db/db mice:
ILB-MCO-0904Q 300 mg/kg: Did not show significant effect on plamsa glucose levels with acute or chronic administration.
ILB-MCO-0905Q 300 mg/kg: Significant lowering of plasma glucose levels 3 hrs post dosing with single dose. Chronic treatment (21 days) did not show significant reduction in plasma glucose levels but showed significant reduction in plasma insulin levels. This, followed by compound administration on day 21, showed significant lowering of plasma glucose levels.
ILB-MCO-0906Q 300 mg/kg: Significant lowering of plasma glucose levels 3 hrs post dosing with single dose. Chronic treatment (21 days) did not show significant reduction in plasma glucose levels. This, followed by compound administration on day 21, showed significant lowering of plasma glucose levels.
ILB-MCO-0904Q, ILB-MCO-0905Q and ILB-MCO-0906Q showed 15%, 13% and 11% reduction in plasma cholesterol levels respectively, after 21 days treatment post 4 hrs fasting, without dosing on the day.
ILB-MCO-0904Q, ILB-MCO-0905Q and ILB-MCO-0906Q showed 21%, 27% and 28% reduction in plasma triglyceride levels respectively, after 21 days treatment post 4 hrs fasting, without dosing on the day.
Animal number 5 belongs to control group was found dead on 03.08.09 and was examined by necropsy. This animal lost around 4.5 g body weight from 30.07.09 to 03.08.09. The exact cause of animal death could not be ascertained. However, there were no significant changes in body weight with test compounds administration compared to control group.

We claim:

1. Synergistic pharmaceutical co-crystal composition comprising Quercetin and an anti-diabetic agent(s).

2. The pharmaceutical co-crystal composition as claimed in claim 1, wherein said anti-diabetic agent is selected from biguanide group like metformin; sulfonylurea group like tolazamide, glipizide, glimepiride; Alpha-glucosidase inhibitors (acarbose); members of the thiazolidinedione class such as rosiglitazone, pioglitazone and miglitol-a glucosidase inhibitor.

3. The pharmaceutical co-crystal composition as claimed in claim 1, wherein said anti-diabetic agent is Metformin, its pharmaceutical salts or its polymorphs.

4. The pharmaceutical co-crystal composition as claimed in claim 1, wherein said Quercetin can be selected in the form of hydrates or its polymorphs.

5. The pharmaceutical co-crystal composition as claimed in claim 1, wherein said composition further comprises one or more suitable pharmaceutical diluent, carriers or excipient.

6. A process for preparing pharmaceutical co-crystal composition as in claim 1, comprises:
(a) obtaining pharmaceutical co-crystal of Quercetin and Metformin or its salts by reaction crystallization or by melting or by mixing, and grinding the same in 1:1 to 1: 3 ratio respectively and
(b) incorporating the co-crystals thus obtained in step (a) into a pharmaceutical composition using one or more suitable diluents, carriers or excipient.

7. The process as claimed in claim 6, wherein the mixing and grinding is done in presence or absence of solvent(s).

8. Method for treating metabolic syndrome associated with Hypertension, Diabetic conditions inclusive of obesity and hyperlipidemia, wherein said method comprises administering ‘an effective amount’ of the ‘composition of the invention’ as in claim 1, to the subject suffering from metabolic syndrome associated with Hypertension, diabetic conditions inclusive of obesity and hyperlipidemia.

9. The method as claimed in claim 7, wherein said subject is human.

10. Use of synergistic pharmaceutical co-crystal composition of Quercetin and anti-diabetic agent as in claim 1, in preparing the medicament intended to treat metabolic syndrome associated with Hypertension, diabetic conditions inclusive of obesity and hyperlipidemia.
